(19) United States
(12) Patent Application Publication
(10) Pub. No.: US 2017/0326054 A1
(43) Pub. Date: Nov. 16, 2017

(54) COMPOSITIONS AND METHODS FOR REDUCING OR PREVENTING DENTAL CALCULUS ACCUMULATION IN COMPANION ANIMALS

(71) Applicant: Nestec SA, Vevey (CH)

(72) Inventors: Julie K. Spears, St. Louis, MO (US); Amy Alderton, St. Louis, MO (US); Peifang Zhang, Chesterfield, MO (US); Robert Miller, St. Louis, MO (US)

(21) Appl. No.: 15/591,852

(22) Filed: May 10, 2017

Related U.S. Application Data
(60) Provisional application No. 62/334,787, filed on May 11, 2016.
(60) Provisional application No. 62/436,751, filed on Dec. 20, 2016.

(51) Int. Cl. A61K 8/44; A61K 8/27; A61K 8/368; A23K 20/20; A23K 20/22; A23K 20/10; A23K 50/40; A61Q 11/00; A61K 8/362

(52) U.S. Cl. CPC A61K 8/44; A61Q 11/00; A61K 8/27; A61K 8/368; A61K 8/362; A23K 20/22; A23K 20/10; A23K 50/40; A23K 20/30; A61K 2800/524

(57) ABSTRACT

A method of reducing or preventing conversion of plaque to calculus in a companion animal. The method includes orally administering to the companion animal a composition comprising a zinc salt and one or more chelating agents. The zinc salt and/or the chelating agents can be present in an amount from about 0.01% to about 5.0% of the composition.

### CROSS REFERENCE TO RELATED APPLICATIONS

This application claims priority to U.S. Provisional Application Ser. No. 62/334,787 filed May 11, 2016, and to U.S. Provisional Application Ser. No. 62/436,751 filed Dec. 20, 2016, the disclosures of which are both incorporated in their entireties herein by this reference.

### BACKGROUND

Dental disease is a common problem in dogs and cats and is becoming even more prevalent as the pet population ages. According to the American Veterinary Dental Society, 80% of dogs and 70% of cats show signs of periodontal disease by the age of three years, and 85% of all adult pets have periodontal disease.

Periodontal disease begins when bacteria in the mouth form a substance called plaque that adheres to the surface of the teeth. Subsequently, minerals in the saliva harden the plaque into dental calculus, also known as tartar, which is firmly attached to the teeth.

Plaque and calculus can spread to under the gum line (“sub-gingival”). Bacteria in the sub-gingival plaque can damage the supporting tissues around the tooth, eventually leading to loss of the tooth. Sub-gingival bacteria secrete toxins which contribute to the tissue damage if untreated. These bacteria also stimulate the immune system of the animal such that white blood cells and inflammatory chemical signals move into the periodontal space between the gum or bone and the tooth. The white blood cells destroy the bacteria but also release chemicals that damage the supporting tissues of the tooth, especially when there is severe accumulation of plaque and calculus.

Periodontal disease includes gingivitis (inflammation of the gums) and periodontitis (loss of bone and soft tissue around the teeth). Effects within the oral cavity include damage to or loss of gum tissue and bone around the teeth, development of a hole (‘fistula’) from the oral cavity into the nasal passages causing nasal discharge, fractures of the jaw following weakening of the jaw bone, and bone infection (‘osteomyelititis’). Bacteria from the mouth can enter the bloodstream and be carried through the body; studies in dogs have shown that periodontal disease is associated with microscopic changes in the heart, liver and kidneys.

### SUMMARY

The present disclosure relates generally to compositions comprising increased amounts of disodium EDTA, sodium citrate, and zinc salts, and relates to methods comprising administering the compositions to a dog or a cat. The composition can be provided in drinking water, a foam, a treat, an extruded kibble, a coating of a pet food, an edible film, or a wet pet food, for example, retorted pet food or retorted canned pet food.

Known pet products intended to promote oral health by reducing plaque and calculus accumulation rely on mechanical removal of plaque and tartar. The present inventors recognized that the hard-texture of treats by itself is not completely effective in preventing periodontal disease in companion animals and thus sought to minimize and/or prevent calcification of plaque to calculus and neutralize breath malodor by incorporating a non-mechanical approach to such pet products.

In this regard, the present inventors noted that commercially available compositions used as a water additive do not significantly improve calculus status. The present inventors found that a blend of increased amounts of disodium EDTA dihydrate, trisodium citrate dihydrate, and zinc chloride reduced calculus by 20% compared to control (tap water that was also statistically similar to the commercial compositions) (P<0.05). The methods and compositions disclosed herein improved the oral health benefit compared to commercial compositions beyond mechanical action alone because the blend prevents calcification of plaque to calculus. At the same time, there was an improvement in oral health measures without increasing caloric consumption, and the plaque biofilm could be more easily removed via mechanical chews or brushing. The methods and compositions disclosed herein also improved removal of tartar as the tartar became softer and easier to displace.

Without being bound by theory, the present inventor believe that chelating agents, such as disodium EDTA dihydrate and trisodium citrate dihydrate, are effective against oral disease-associated bacteria; and a zinc salt, such as zinc chloride or zinc citrate, reduces volatile sulfur compounds by oxidizing thiol groups in precursors of volatile sulfur compounds as well as acting as an anti-microbial.

Accordingly, in a general embodiment, a method of reducing or preventing conversion of plaque to calculus in a companion animal is provided. The method comprises orally administering to the companion animal a composition comprising one or more chelating agents selected from the group consisting of sodium citrate, potassium citrate, zinc citrate, disodium ethylenediaminetetraacetic acid (EDTA), tetrasodium EDTA, ethylene glycol tetraacetic acid (EGTA), diethylenetriaminepentaacetic acid (DTPA), ethylenediamine-N,N′-bis(2-hydroxyphenylacetic acid) (EDDHA), iminodiacetic acid (IDA), trans-1,2-diaminocyclohexane-N,N,N′,N′-tetraacetate (CDTA), hydroxyethyenediaminetriacetic acid (HEDTA), 2-hydroxyethyliminodiacetic acid (HEIDA), and nitrilotriacetic acid (NTA). In one embodiment, the one or more chelating agents can be present in a total amount of at least about 6.0 g/L of the composition, and the composition further comprises a zinc salt. In another embodiment, the chelating agents can be present from about 0.01% to about 5.0% of the composition. Additionally, the zinc salt can be present from about 0.01% to about 5.0% of the composition. In one embodiment, the zinc citrate can be zinc citrate trihydrate. In another embodiment, the disodium ethylenediaminetetraacetic acid (EDTA) can be calcium disodium EDTA.

In one embodiment, the method can also reduce or prevent breath malodor.

In another embodiment, the one or more chelating agents is about 6.0 g/L to about 40.0 g/L, alternatively about 6.0 g/L to about 35.0 g/L, alternatively about 6.0 g/L to about 30.0 g/L, alternatively about 6.0 g/L to about 25.0 g/L, alternatively about 6.0 g/L to about 20.0 g/L, or alternatively about 6.0 g/L to about 15.0 g/L of the composition. In an embodiment, the total amount of the one or more chelating agents is at least about 8.0 g/L of the composition. In another embodiment, the one or more chelating agents is about 8.0 g/L to about 40.0 g/L, alternatively about 8.0 g/L to about 35.0 g/L, alternatively about 8.0 g/L to about 30.0 g/L, alternatively about 8.0 g/L to about 25.0 g/L, alternatively about 8.0 g/L to about 20.0 g/L, or alternatively about 8.0 g/L to about 15.0 g/L of the composition.

In an embodiment, the chelating agent comprises (i) a first chelating agent selected from the group consisting of sodium citrate, potassium citrate, zinc citrate and mixtures thereof and (ii) a second chelating agent selected from the group consisting of disodium ethylenediaminetetraacetic acid (EDTA), tetrasodium EDTA, ethylene glycol tetraacetic acid (EGTA), diethylenetriaminepentaacetic acid (DTPA), ethylenediamine-N,N′-bis(2-hydroxyphenylacetic acid) (EDDHA), iminodiacetic acid (IDA), trans-1,2-diaminocyclohexane-N,N,N′,N′-tetraacetate (CDTA), hydroxyethyenediaminetriacetic acid (HEDTA), 2-hydroxyethyliminodiacetic acid (HEIDA), nitrilotriacetic acid (NTA) and mixtures thereof.

The first chelating agent can be present in a total amount of at least about 6.0 g/L of the composition. In another embodiment, the first chelating agent is about 6.0 g/L to about 30.0 g/L, alternatively about 6.0 g/L to about 25.0 g/L, alternatively about 6.0 g/L to about 20.0 g/L, or alternatively about 6.0 g/L to about 15.0 g/L of the composition. In one embodiment, the first chelating agent can comprise trisodium citrate dihydrate.

The second chelating agent can be present in a total amount of at least about 2.0 g/L of the composition. In another embodiment, the second chelating agent is about 2.0 g/L to about 10.0 g/L, alternatively about 2.0 g/L to about 8.0 g/L, or alternatively about 2.0 g/L to about 5.0 g/L of the composition. In one embodiment, the second chelating agent can comprise disodium EDTA dihydrate.

In an embodiment, the zinc salt is selected from the group consisting of zinc chloride, zinc lactate, zinc gluconate, zinc citrate, and mixtures thereof. The zinc salt can be present in a total amount of at least about 0.075 g/L of the composition. In another embodiment, the zinc salt is about 0.075 g/L to about 0.375 g/L, alternatively about 0.075 g/L to about 0.300 g/L, alternatively about 0.075 g/L to about 0.225 g/L, or alternatively about 0.075 g/L to about 0.150 g/L of the composition. In one embodiment, the zinc salt can comprise zinc chloride.

In an embodiment, the composition further comprises a preservative. The preservative can comprise a benzoate salt, tocopherols, (for example, vitamin E), ascorbic acid and its derivatives (for example, vitamin C and ascorbyl palmitate).

In an embodiment, the composition further comprises citric acid.

In an embodiment, the composition is administered in an orally acceptable carrier selected from the group consisting of water, a foam, a pet treat, an extruded kibble, a coating of a pet food, an edible film, a wet pet food, a retorted pet food, and a retorted canned pet food.

In an embodiment, the composition is administered without increasing the daily caloric intake of the companion animal relative to a time period comprising at least one week immediately prior to the administering.

In another embodiment, the present disclosure provides a comestible composition comprising one or more chelating agents selected from the group consisting of sodium citrate, potassium citrate, zinc citrate, disodium ethylenediaminetetraacetic acid (EDTA), tetrasodium EDTA, ethylene glycol tetraacetic acid (EGTA), diethylenetriaminepentaacetic acid (DTPA), ethylenediamine-N,N′-bis(2-hydroxyphenylacetic acid) (EDDHA), iminodiacetic acid (IDA), trans-I,2-diaminocyclohexane-N,N,N′,N′-tetraacetate (CDTA), hydroxyethyenediamine triacetic acid (HEDTA), 2-hydroxyethyliminodiacetic acid (HEIDA), and nitrilotriacetic acid (NTA), the one or more chelating agents are present in a total amount of at least about 6.0 g/L of the comestible composition. Additionally, the comestible composition can further comprise a zinc salt.

In an embodiment, the one or more chelating agents comprise trisodium citrate dihydrate in an amount of at least about 6 g/L of the composition and disodium EDTA dihydrate in an amount of at least about 2 g/L of the composition, and the zinc salt comprises zinc chloride in an amount of at least about 0.075 g/L of the composition. In an embodiment, the one or more chelating agents comprise trisodium citrate dihydrate in an amount of at least about 6 g/L to about 30 g/L of the composition and disodium EDTA dihydrate in an amount of at least about 2 g/L to about 10 g/L of the composition, and the zinc salt comprises zinc chloride in an amount of at least about 0.075 g/L to about 0.375 g/L of the composition.

An advantage of one or more embodiments provided by the present disclosure is to go beyond mechanical action to slow calcification of plaque to calculus in a companion animal.

Another advantage of one or more embodiments provided by the present disclosure is improve oral health without increasing calories consumed in a companion animal.

Yet another advantage of one or more embodiments provided by the present disclosure is to allow plaque biofilm to be easily removed via mechanical chews or toothbrushing by preventing its conversion to calculus which can only be removed by professional cleaning.

Still another advantage of one or more embodiments provided by the present disclosure is a positive benefit on oral health without impacting other health parameters or food and water intake.

Additional features and advantages are described herein and will be apparent from, the following Detailed Description and the Figures.

### BRIEF DESCRIPTION OF THE FIGURES

FIG. 1 is a table containing group allotment details for the study disclosed herein.

FIG. 2 is a graph of gingival index as measured in the study disclosed herein.

FIG. 3 is a graph of tartar score as measured in the study disclosed herein.

FIG. 4 is a graph of average daily food intake as measured in the study disclosed herein.

FIG. 5 is a graph of average daily water intake as measured in the study disclosed herein.

FIG. 6 shows tartar index reduction as measured in the study disclosed herein.

### DETAILED DESCRIPTION

### Definitions

Some definitions are provided hereafter. Nevertheless, definitions may be located in the “Embodiments” section below, and the above header “Definitions” does not mean that such disclosures in the Preferred Embodiments” section are not definitions.

As used in this disclosure and the appended claims, the singular forms “a,” “an” and “the” include plural referents unless the context clearly dictates otherwise. Thus, for example, reference to “an ingredient” or “the ingredient” includes two or more ingredients. The term “and/or” used in the context of “X and/or Y” should be interpreted as “X,” or “Y,” or “X and Y.” Where used herein, the term “example,” particularly when followed by a listing of terms, is merely exemplary and illustrative, and should not be deemed to be exclusive or comprehensive.

As used herein, “about” is understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. A range that is “between” two values includes those two values. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight of the total weight of the composition unless expressed otherwise. When reference is made to the pH, values correspond to pH measured at 25° C. with standard equipment.

The terms “food,” “food product” and “food composition” mean a product or composition that is intended for ingestion by an animal and provides at least one nutrient to the animal. The term “pet food” means any food composition intended to be consumed by a pet. The term “pet” means any animal which could benefit from or enjoy the compositions provided by the present disclosure. For example, the pet can be an avian, bovine, canine, equine, feline, hircine, lupine, murine, ovine, or porcine animal, but the pet can be any suitable animal. The term “companion animal” means a dog or a cat.

A “dry” food composition has less than 10% moisture and/or a water activity less than 0.64, or both. A “semi-moist” food composition has 11% to 20% moisture and/or a water activity of 0.64 to 0.75, or both. A “wet” food composition has more than 20% moisture and/or a water activity higher than 0.75, or both.

“Kibbles” means pieces of dry or semi-moist pet food which can have a pellet shape or any other shape. Non-limiting examples of kibbles include particulates; pellets; pieces of pet food, dehydrated meat, meat analog, vegetables, and combinations thereof; and pet snacks, such as meat or vegetable jerky, rawhide, and biscuits.

The term “biofilm” refers to microorganisms attached to surfaces and the subsequent development multiple layers of cells. The term “dental caries” refers to a localized destruction of tissues of a tooth by acid produced from bacterial degradation of fermentable sugars. The etiological agent of dental caries is typically S. mutans. Degradation of fermentable sugars by S. mutans on the tooth surface produces an acid that destroys oral tissues, and more particularly, enamel and dentin. The term “plaque” is a general term for the diverse microbial community (predominantly bacteria) found on the tooth surface, embedded in a matrix of polymers of bacterial and salivary origin. The term “gingivitis” refers to inflammation of gingival tissue without loss of connective tissue. The term “oral diseases” refers to diseases and disorders affecting the oral cavity or associated medical conditions. Oral diseases include, but are not limited to, dental caries; and periodontal diseases (e.g., gingivitis, adult periodontitis, and early-onset periodontitis). The term “periodontal disease” refers to an inflammatory process of the gingival tissues and/or periodontal membrane of the teeth, resulting in a deep gingival sulcus, possibly producing periodontal pockets and loss of alveolar bone. The term “periodontitis” refers to inflammation and loss of connective tissue of the supporting or surrounding structure of teeth with loss of attachment.

The term “prophylaxis” or “prevention” refers to at least preventing a condition associated with oral bacteria occurring in a mammal, particularly when the mammal is found to be predisposed to having the condition but has not yet been diagnosed as having it.

The term “treatment” refers to an intervention performed with the intention of preventing the further development or altering the pathology of an existing disorder. Accordingly, “treatment” refers to both therapeutic treatment and prophylactic or preventative measures. Those in need of treatment include those already with the disorder as well as those in which the disorder is to be prevented. “Treatment” includes, but is not limited to, modulating the condition, inhibiting the condition and/or alleviating the condition.

The term “antimicrobial” refers to a compound or a composition that kills or inhibits the growth of microorganisms, such as bacteria and yeasts.

The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified.

Any embodiment disclosed herein can be combined with any other embodiment disclosed herein.

The term “sodium EDTA” refers to any of the sodium salts of ethylenediaminetetraacetic acid, and includes monosodium EDTA, disodium EDTA, trisodium EDTA and tetrasodium EDTA. The term “sodium EDTA” also includes the hydrates of any of the sodium salts of ethylenediaminetetraacetic acid.

The term “sodium citrate” refers to any of the sodium salts of citric acid, and includes monosodium citrate, disodium citrate, and trisodium citrate. The term “sodium citrate” also includes the hydrates of any of the sodium salts of citric acid.

The term “zinc citrate” refers to any hydrate forms of zinc citrate.

### Embodiments

An aspect of the present disclosure is a method of reducing or preventing conversion of plaque to calculus in a companion animal is provided. The method can treat or prevent oral diseases such as dental caries, gingivitis, periodontitis, and oral bacterial infections or diseases.

The method comprises orally administering to the companion animal a composition comprising one or more chelating agents selected from the group consisting of sodium citrate, potassium citrate, zinc citrate, disodium ethylenediaminetetraacetic acid (EDTA), tetrasodium EDTA, ethylene glycol tetraacetic acid (EGTA), diethylenetriaminepentaacetic acid (DTPA), ethylenediamine-N,N′-bis(2-hydroxyphenylacetic acid) (EDDHA), iminodiacetic acid (IDA), trans-I,2-diaminocyclohexane-N,N,N′,N′-tetraacetate (CDTA), hydroxyethyenediamine triacetic acid (HEDTA), 2-hydroxyethyliminodiacetic acid (HEIDA), and nitrilotriacetic acid (NTA), the one or more chelating agents are present in a total amount of at least about 6.0 g/L of the composition, and the composition further comprises a zinc salt. In an embodiment, the total amount of the one or more chelating agents is at least about 8.0 g/L to about 25.0 g/L of the composition. In another embodiment, the amount of the zinc salt is at least about 0.075 g/L to about 0.150 g/L of the composition.

In another embodiment, the chelating agents can be present from about 0.01% to about 5.0% of the composition. In one aspect, the amount can be from about 0.01% to about 3.0%. In another aspect, the amount can be from about 0.01% to about 1.0%. Other amounts of the chelating agents can include from about 0.01% or about 0.1% to about 0.5%, about 0.6%, about 0.7%, about 0.8%, or even about 0.9% of the composition. Additionally, the zinc salt can be present from about 0.01% to about 5.0% of the composition. In one aspect, the amount can be from about 0.01% to about 3.0%. In another aspect, the amount can be from about 0.01% to about 1.0%. Other amounts of the zinc salt can include from about 0.01% or about 0.1% to about 0.5%, about 0.6%, about 0.7%, about 0.8%, or even about 0.9% of the composition. These amounts are useful for the present methods and compositions.

Another aspect of the present disclosure is the composition that can be utilized in this method. The composition can be a comestible composition in an orally acceptable carrier selected from the group consisting of water, a foam, a pet treat, an extruded kibble, a coating of a pet food, an edible film, a wet pet food, a retorted pet food and a retorted canned pet food. The composition may further comprise one or more ingredients selected from the group consisting of water, a buffer, a stabilizing agent, a suspending agent, a thickening agent, a gelling agent, a flavoring agent, an essential amino acid, a non-essential amino acid, a carbohydrate, a lipid, a vitamin, a mineral (e.g., a mineral salt), an antioxidant, a herbal, a surfactant, an antimicrobial compound (e.g., an antimicrobial peptide), and a pH adjuster (e.g., citric acid).

Generally, the chelating agent can comprise (i) a first chelating agent selected from the group consisting of sodium citrate, potassium citrate, zinc citrate and mixtures thereof and (ii) a second chelating agent selected from the group consisting of disodium ethylenediaminetetraacetic acid (EDTA), tetrasodium EDTA, ethylene glycol tetraacetic acid (EGTA), diethylenetriaminepentaacetic acid (DTPA), ethylenediamine-N,N′-bis(2-hydroxyphenylacetic acid) (EDDHA), iminodiacetic acid (IDA), trans-I,2-diaminocyclohexane-N,N,N′,N′-tetraacetate (CDTA), hydroxyethyenediaminetriacetic acid (HEDTA), 2-hydroxyethyliminodiacetic acid (HEIDA), nitrilotriacetic acid (NTA) and mixtures thereof.

In a one embodiment, the first chelating agent comprises trisodium citrate dihydrate, and the second chelating agent comprises disodium EDTA dihydrate. The first chelating agent (e.g., trisodium citrate dihydrate) can be present in a total amount of at least about 6 g/L of the composition. The second chelating agent (e.g., disodium EDTA dihydrate) can be present in a total amount of at least about 2 g/L of the composition.

In an embodiment, the zinc salt is selected from the group consisting of zinc chloride, zinc lactate, zinc gluconate, and mixtures thereof. The zinc salt (e.g., zinc chloride) can be present in a total amount of at least 0.075 g/L of the composition.

In one specific aspect, the composition comprises a blend of 2 g/L disodium EDTA dihydrate, 6.4 g/L trisodium citrate dihydrate, and 0.078 g/L zinc chloride.

In an embodiment, the composition further comprises a preservative, for example a benzoate salt.

The composition can be administered to the companion animal in drinking water and/or in feed once a day or multiple times per day. In an embodiment, the composition is administered without increasing the daily caloric intake of the companion animal relative to a time period comprising at least one week immediately prior to the administering, in one aspect, at least two weeks, in another aspect, at least three weeks, and in one specific aspect, at least one month.

The composition can comprise an antibiotic. Non-limiting examples of suitable antibiotics include β-lactam inhibitors (e.g., penicillin, ampicillin, amoxicillin, and methicillin), cephalosporins (e.g., cephalothin and cephamycin), aminoglycosides (e.g., streptomycin and tobramycin), polyenes (e.g., amphotericin and nystatin), macrolides (e.g., erythromycin), tetracyclines (e.g., tetracycline and doxycycline), nitroimidazole (e.g., metronidazole), quinolones (e.g., nalidixic acid), rifamycins (e.g., rifampin), sulfonamides (e.g., sulfanilamide), nitroaromatics (e.g., chloramphenicol), pyridines (e.g., isoniazid) and mixtures thereof.

The composition can comprise a polyphenol. Non-limiting examples of suitable polyphenols include epigallocatechin gallate (EGCg) and also as tannins from thyme, cashew and/or eucalyptus. The composition can comprise a polyol which can limit growth and biofilm formation in oral Streptococci that are associated with dental plaque, the precursor of calculus (tartar) and dental caries. Non-limiting examples of suitable polyols include sorbitol, mannitol, maltitol, and mixtures thereof.

The composition can comprise a bacteriocin. Non-limiting examples of suitable bacteriocins include nisin, epidernin, gallidermin, cinnamycin, duramycin, lacticin 481, mutacin I, B-Ny266, mutacin 1140, and mixtures thereof. The composition can comprise an antiseptic which can kill or inhibit the growth of microorganisms on the external surfaces of the body. Non-limiting examples of suitable antiseptics include triclosan, chlorhexidine salt, cetylpyridinium chloride, and mixtures thereof.

The composition can comprise an antibiofilm compound. Non-limiting examples of suitable antibiofilm compounds include DNase I, Proteinase K, apyrase, cis-2-decenoic acid, alginate lyase, lactoferrin, gallium, 5-fluorouracil, and mixtures thereof.

The composition can comprise an anti-caries agent. Non-limiting examples of suitable anti-caries agents include benzoic esters, sesquiterpene alcohols (e.g., farnesol, nerolidol, bisabolol and santalol), halogenated carbanilides, phenolic compounds, aromatic halophenols, resorcinols, catechols, bisphenolic compounds, histidine-rich polypeptides, fluorides (sodium fluoride, stannous fluoride, amine fluorides, and monosodiumfluorophosphate), calcium lactate, calcium glycerophosphate, proline-rich proteins, a non-immunogenic amino acid segment, antibodies of S. mutans, and mixtures thereof.

### EXAMPLE

By way of example and not limitation, the following non-limiting example study is illustrative of compositions and methods for reducing plaque and calculus accumulation in companion animals in one or more embodiments provided by the present disclosure.

### Example 1. Feeding 1× Additive, 2× Additive and TROPICLEAN®

The objective of the study was to evaluate the effect of water drops on oral health (plaque, tartar and gingival inflammation) in pets. Specifically, the efficacy of two different concentrations of a water additive were compared to a commercial water additive (TROPICLEAN®, an additive that can comprise citric acid, cetylpyridium chloride, chlorophyllin, glycerin, potassium sorbate, omega 3 fatty acids, omega 6 fatty acids, and green tea leaf extract) on oral health in dogs (plaque, gingival, and calculus index, breath malodor, and oral microbiota).

Thirty dogs (one year and older) were selected and fed in an incomplete crossover design with a standard adult dog food and tap water (control) or tap water containing 1) a blend of 1 g/L disodium EDTA dihydrate, 3.2 g/L trisodium citrate dihydrate, and 0.039 g/L zinc chloride (“1× Additive”); 2) a blend of 2 g/L disodium EDTA dihydrate, 6.4 g/L trisodium citrate dihydrate, and 0.078 g/L zinc chloride (“2× Additive”), or 3) TROPICLEAN® (citric acid, cetylpyridium chloride, chlorophyllin, glycerin, potassium sorbate, omega 3 fatty acids, omega 6 fatty acids, and green tea leaf extract).

Thirty adult beagle dogs (1 year and older) were selected for prescreening. The prescreening consisted of a standard physical exam and a thorough dental exam. For the dental exam, only dogs with no obvious dental problems and all teeth mandated by the Veterinary Oral Health Council (VOHC) were selected to start the trial. Required teeth were as follows: upper jaw: 3^{rd} incisor, canine, 3^{rd} premolar, 4^{th} premolar, and 1^{st} molar; and lower jaw: canine, 3^{rd} premolar, 4^{th} premolar, and 1^{st} molar.

Feeding started in Aug. 11, 2015 and was completed on Dec. 3, 2015. The study was an incomplete crossover design and was established as a VOHC-like protocol as described hereafter:

Phase 1
Day 0: Sedation, Gingival scoring, Teeth cleaning and polishing
Days 1-56: Dogs fed allotted water
Day 56: CBC/blood chemistry, Sedation, Gingival/plaque/calculus scoring,
Breath/plaque collection, Teeth cleaning and polishing
Phase 2
Days 57-112: Dogs fed allotted water
Day 112: CBC/blood chemistry, Sedation, Gingival/plaque/calculus
scoring, Breath/plaque collection
End of Trial

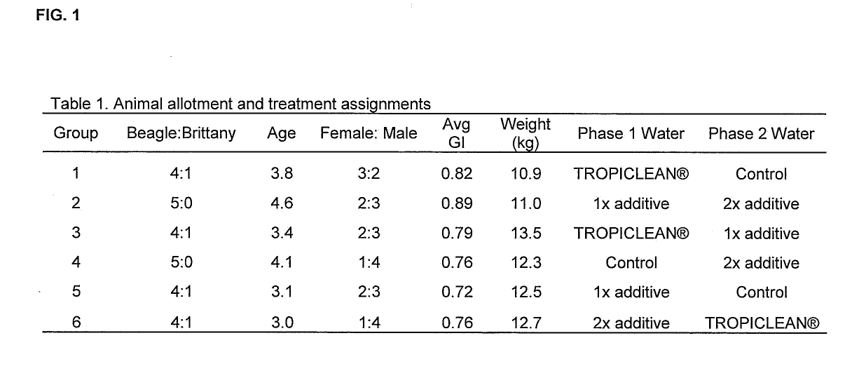

Animals were allotted to six groups of five dogs by breed, age, gender, and average gingival index on Day 0. They were fed their allotted waters ad libitum as described in Table 1 in FIG. 1 for 56 days during each phase. Group allotment details are also shown in this table.

Dogs were anesthetized/sedated three times during this trial. Dogs were sedated for gingival scoring and a complete dental prophylaxis at Days 0 and 56, the beginning of each of the two phases of the trial. Dogs were also sedated for gingival, plaque and calculus scoring at Days 56 and 112, the end of each of the two phases of the trial (sedation on Days 0, 56, and 112). Anesthesia/sedation was required to obtain complete removal of dental substrates at the beginning of the trial and to obtain accurate dental substrate scores at the end of each phase.

Dogs were presented for scoring in random order. A single score representing the worst area of the tooth was assigned for each of the eighteen VOHC designated teeth. Gingival scoring was performed on all dogs according to the following scoring system:

Gingivitis Scoring Method

| Score | Degree of Inflammation |
|---|---|
| 0 | no inflammation or redness |
| 1 | mild inflammation, slight redness or swelling, no |
| | bleeding |
| 2 | moderate inflammation, redness and swelling, bleeds on |
| | gentle probing of sulcus |
| 3 | marked inflammation, red, very swollen margins, |
| | spontaneous bleeding on probing |
| 4 | severe inflammation, bright red, very swollen margins, |
| | profuse bleeding on probing or touching of tissue |

Any dog with an average gingival score greater than 2 was not used. Gingival scoring was followed by a complete dental prophylaxis (cleaning and polishing) on days 0 and 56. Dogs were blocked into 6 groups of 5 dogs and assigned to waters on the basis of gingival score, breed, age, gender, and sex.

After 56 days on each phase (Days 56 and 112), dogs were then sedated for gingival scoring (repeated as listed above), plaque and calculus scoring. Dogs were presented for scoring in random order. After gingival scoring, a 2% erythrosin plaque-disclosing solution was applied to the teeth, rinsed with tap water and a single score for plaque recorded for the gingival halves of the eighteen VOHC-designated teeth. The following plaque scoring method was applied (coverage×thickness):

Plaque Scoring Method

| Score | % of Plaque Coverage |
|---|---|
| 0 | No plaque detected |
| 1 | 1-24 |
| 2 | 25-49 |
| 3 | 50-74 |
| 4 | 75-100 |

| Score | Plaque Thickness | Disclosing Agent Color |
|---|---|---|
| 1 | Light | Pink to Light Red |
| 2 | Medium | Red |
| 3 | Heavy | Dark Red |

Following plaque scoring, samples of plaque were collected with swabs, and samples were frozen at −80° C. until further analysis. Teeth were then brushed to remove plaque, rinsed with tap water, and air dried, and then a single score for calculus recorded for the whole tooth for all eighteen VOHC-designated teeth. The following calculus scoring method was applied (coverage×thickness):

Plaque Scoring Method

| Score | |
|---|---|
| | % of Plaque Coverage |
| 0 | No plaque detected |
| 1 | 1-24 |
| 2 | 25-49 |
| 3 | 50-74 |
| 4 | 75-100 |
| | Plaque Thickness |
| 1 | Thin |
| 2 | Medium |
| 3 | Thick |

Regarding other key measurements, food intake and water intake were measured daily. Blood samples were collected for CBC and blood chemistry analysis at the end of each phase, and a physical exam was performed at the beginning and end of each phase.

### Results

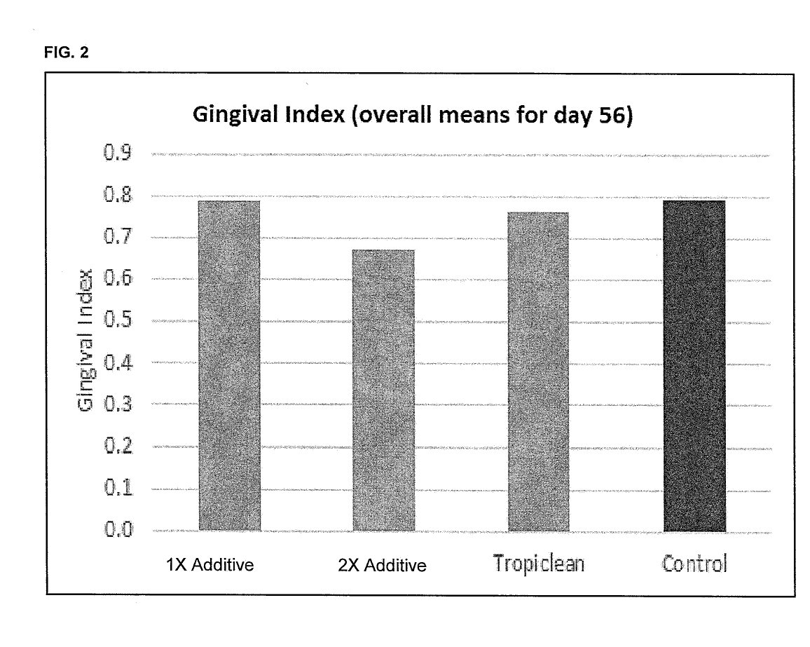

There was no significant difference of gingival index among any of the treatment groups (FIG. 2). Gingival index scores were low, indicating that treatments did not cause an inflammation of gums.

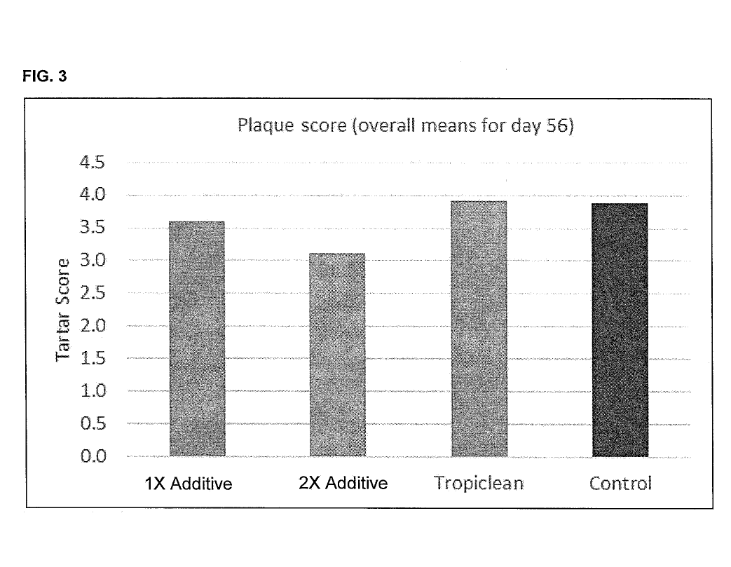

Overall tartar score means for day 56 are shown in FIG. 3. Tartar was significantly decreased (p<0.05) in dogs receiving 2X Additive compared to control and TROPICLEAN® (overall diet means for day 56). Overall percent reduction, calculated as [(Control−2× Additive)/Control]*100 was 19.9%. The reduction in tartar score was increased 2.2% compared to a shorter feeding time. Overall percent reduction from TROPICLEAN®, calculated as [(TROPICLEAN®−2× Additive)/TROPICLEAN®]*100 was 20.7%.

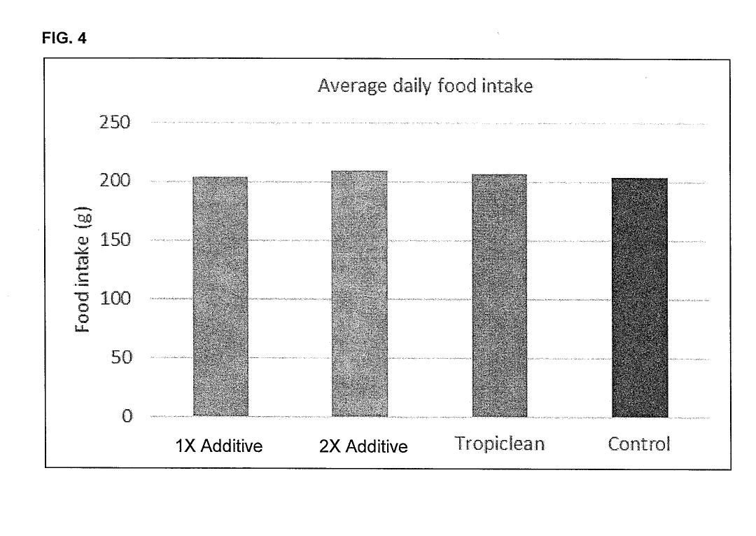

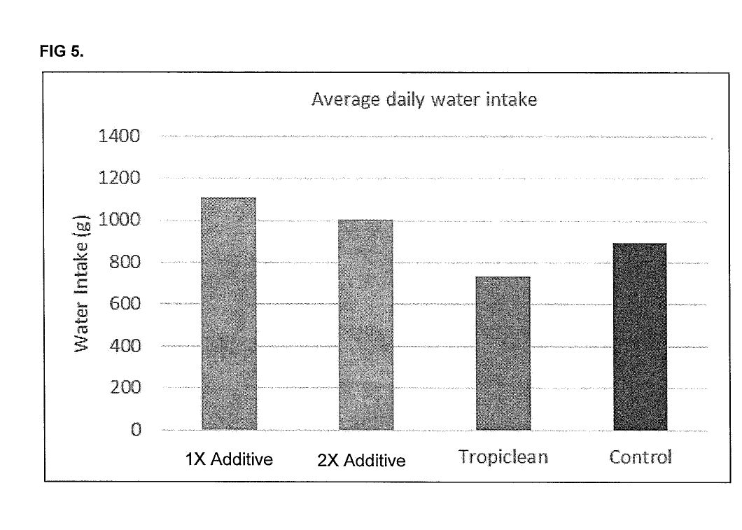

Dogs were fed to maintain an ideal body condition score. There was no significant difference of daily food intake among any of the treatment groups (FIG. 4). Water was offered ad libitum, and daily intake was determined by weight instead of volume. Daily water intake was impacted by treatment (FIG. 5). Dogs consumed significantly less water containing TROPICLEAN® when compared to the 1× Additive treatment. Dogs receiving the Additive treatments in their water consumed numerically more than when offered control.

### Conclusions

Oral health results from this test agreed with previous research. Feeding for a longer period of time increased tartar reduction noted with 2× Additive compared to previous tests. Health parameters remained in the normal range, and water intake was not impacted by addition of Additive. This test demonstrated that consumption of 2× Additive for 56 days has a positive benefit on oral health without impacting other health parameters.

### Example 2. Feeding Treat with 1× Additive

Pre-Test Phase (Day −7 to Day 0): Dogs assigned to this study were on a previous dental study. The previous study included a pre-test phase of seven days that was conducted before the initiation of the treatment period. During the pre-test phase, the dogs selected for this study were weighed and fed the control diet (a kibble that was fed dry). Each animal had its teeth scaled and polished upon initiation of the pre-test phase (Day −7). On Day 0, plaque and gingivitis were evaluated and the teeth were once again cleaned and polished for each animal. The scores obtained were indicative of the rate of plaque buildup for each animal when being fed the control diet only. Animals were stratified into four groups based on plaque scores. This procedure was performed in an attempt to reduce the variability of scores during the test treat phase. Animals remained in the same groups for this study. Following the dental scoring, each animal had a dental cleaning and polishing procedure. After this procedure a disclosing agent of 2% Eosin was applied to all test teeth and an oral examination was performed to ensure there was no remaining plaque or calculus buildup and a clean mouth was used for the start of the study.

Test Phase (Day 0 to Day 63): One hundred (100) beagle dogs, one to seven years of age, were used in the study (25 dogs per group, 50 males and 50 females). Dogs were grouped in such manner to reduce variability among the study groups based on the dental scores in the pre-test phase. On Days 0 through 63 inclusive, all dogs assigned to Group 1 were fed the control diet only. Dogs assigned to Group 2 were offered the control diet and a treat without 1× Additive, dogs assigned to Group 3 were offered the control diet and a treat with 1× Additive in the treat; and dogs assigned to Group 4 were offered the control diet and a treat with 1× Additive as a paste on the treat. On Days 35 and 63, each animal had gingivitis and calculus evaluations by one technician, and a plaque evaluation by a second technician. Each animal's mouth was also inspected for evidence of lacerations or areas of non-gingival inflammation/ulceration.

During the study (Days 0-63), the control diet was supplied to each animal once daily for approximately one (1) hour in appropriate amounts to maintain ideal body condition for each animal. Food consumption was recorded daily throughout the study beginning on Day 0. The daily food ration was offered as early as possible in the morning throughout the study, except on days scheduled for dental cleaning/scoring (Days 0, 35 and 63), when dogs were fed after the dental cleaning procedure was completed. During the Test Phase (Days 0-63), dogs assigned to treat groups (Group 2, Group 3 and Group 4) were offered one dental test treat, once every day, starting on Day 1. The paste was spread of top of the test treat offered to Group 4 dogs. Treats were weighed before and after offering and treat consumption was recorded.

For the dental scoring and/or cleaning procedures on Days 0, 35 and 63, each animal received anesthetic to maximize comfort during gum and teeth inspection. In the pre-test phase each animal had its teeth scaled and polished on Day 0. Dental cleanings/polishing were performed using general anesthesia. Teeth examined on Days 0, 35 and 63 included the buccal surfaces and both sides of the mouth. Review was limited to these nine teeth: upper jaw—incisor 3, canine (C), premolar 3, premolar 4 and molar 1; lower jaw—canine (C), premolar 3, premolar 4, molar 1 Gingivitis was evaluated followed by calculus accumulation. Plaque was evaluated last after a disclosing agent (2% eosin) was applied to the teeth. Oral scoring was performed by technicians who are approved for scoring VOHC trials.

Gingivitis was assessed on Days 35 and 63. Gingivitis, defined as the inflammation of the gums surrounding the teeth, was evaluated by assigning each tooth a numerical score based on the degree of inflammation. The sum of the teeth scores was divided by the number of teeth examined (18) to obtain a whole mouth mean gingivitis score for each animal.

Gingivitis Scoring Method

| Score | Degree of Inflammation |
|---|---|
| 0 | Absence of inflammation |
| 1 | Mild inflammation; slight change in color, little change in |
| | texture of any portion of the marginal or papillary gingival unit |
| 2 | Mild inflammation; criteria as above but involving the entire |
| | marginal or papillary gingival unit |
| 3 | Moderate inflammation; glazing, redness, edema, and/or |
| | hypertrophy of the marginal or papillary gingival unit |
| 4 | Severe inflammation; marked redness, edema and/or |
| | hypertrophy of the marginal or papillary gingival unit, |
| | spontaneous bleeding, congestion, or ulceration |

Calculus was scored on Days 35 and 63. Calculus was scored quantitatively. Each tooth was assigned a numerical coverage score based on the percentage of calculus coverage and a thickness score (see calculus scoring method below). The score for each tooth was calculated by multiplying the coverage and thickness scores. The sum of the teeth scores was divided by the number of teeth examined (18) to obtain a whole mouth mean calculus score for each animal.

Calculus Scoring Method

| Score | |
|---|---|
| | % of Calculus Coverage |
| 0 | No calculus detected |
| 1 | 1-24 |
| 2 | 25-49 |
| 3 | 50-74 |
| 4 | 75-100 |
| | Calculus Thickness |
| 1 | Thin |
| 2 | Medium |

Plaque was scored on Days 35 and 63. Plaque was evaluated by the extent of plaque and plaque thickness was determined by placing a disclosing agent on the teeth and rinsing the excess off with water. The teeth were visually halved horizontally into gingival and occlusal halves. The gingival half was scored for the percentage of coronal surface covered with plaque and thickness of plaque. The score for each tooth was calculated by multiplying the coverage and thickness scores. The sum of the tooth scores was divided by the number of teeth examined (18) to obtain a whole mouth mean plaque score for each animal.

Plaque Scoring Method

| Score | % of Plaque Coverage |
|---|---|
| 0 | No plaque detected |
| 1 | 1-24 |
| 2 | 25-49 |
| 3 | 50-74 |
| 4 | 75-100 |

| Score | Plaque Thickness | Disclosing Agent Color |
|---|---|---|
| 1 | Light | Pink to Light Red |
| 2 | Medium | Red |
| 3 | Heavy | Dark Red |

Dogs were housed in cages of a size in accordance with the Animal Welfare Act. Lighting and Temperature was kept within conditions in accordance with the Animal Welfare Act. Cages and feeders were cleaned daily and sanitized in accordance with the Animal Welfare Act. Fresh tap water, fit for human consumption, was available ad libitum by means of an automatic watering system. There were no known contaminants that were reasonably expected to be present in the dietary material that were known to be capable of interfering with the purpose or conduct of the study.

In Tables 2 to 7 below, * is t-Test statistically significant (P<0.05) compared to Group 1 (Control Diet); and ** is t-Test statistically significant (P<0.01) compared to Group 1 (Control Diet).

TABLE 2
Day 35 Plaque Reduction Comparisons

| | | Control Diet | % Reduction |
|---|---|---|---|
| | Treat - no Additive | | |
| Mean Left | 5.6 | 6.8 | 17.8** |
| Mean Right | 6.0 | 6.8 | 11.2* |
| Mean Mouth | 5.8 | 6.8 | 14.5** |
| | Treat - 1X Additive in | | |
| | treat (mixed) | | |
| Mean Left | 5.4 | 6.8 | 20.8** |
| Mean Right | 5.6 | 6.8 | 17.1** |
| Mean Mouth | 5.5 | 6.8 | 19.0** |
| | Treat - 1X Additive on | | |
| | treat (paste) | | |
| Mean Left | 5.7 | 6.8 | 15.9** |
| Mean Right | 6.0 | 6.8 | 10.9* |
| Mean Mouth | 5.9 | 6.8 | 13.4** |

TABLE 3
Day 35 Gingivitis Reduction Comparisons

| | | Control Diet | % Reduction |
|---|---|---|---|
| | Treat - no Additive | | |
| Mean Left | 0.1 | 0.2 | 39.5 |
| Mean Right | 0.1 | 0.2 | 56.7* |
| Mean Mouth | 0.1 | 0.2 | 49.7* |
| | Treat - 1X Additive | | |
| | in treat (mixed) | | |
| Mean Left | 0.1 | 0.2 | 81.6** |
| Mean Right | 0.1 | 0.2 | 45.9 |
| Mean Mouth | 0.1 | 0.2 | 60.4* |
| | Treat - 1X Additive | | |
| | on treat (paste) | | |
| Mean Left | 0.1 | 0.2 | 71.1** |
| Mean Right | 0.1 | 0.2 | 51.3 |
| Mean Mouth | 0.1 | 0.2 | 59.3* |

TABLE 4
Day 35 Calculus Reduction Comparisons

| | | Control Diet | % Reduction |
|---|---|---|---|
| | Treat - no Additive | | |
| Mean Left | 1.1 | 1.9 | 42.5** |
| Mean Right | 1.2 | 2.0 | 43.5** |
| Mean Mouth | 1.1 | 2.0 | 43.0** |
| | Treat - 1X Additive in | | |
| | treat (mixed) | | |
| Mean Left | 0.7 | 1.9 | 62.2** |
| Mean Right | 0.8 | 2.0 | 60.2** |
| Mean Mouth | 0.8 | 2.0 | 61.2** |
| | Treat - 1X Additive on | | |
| | treat (paste) | | |
| Mean Left | 0.8 | 1.9 | 58.1** |
| Mean Right | 0.8 | 2.0 | 60.2** |
| Mean Mouth | 0.8 | 2.0 | 59.2** |

TABLE 5
Day 63 Plaque Reduction Comparisons

| | | Control Diet | % Reduction |
|---|---|---|---|
| | Treat - no Additive | | |
| Mean Left | 6.3 | 7.1 | 11.8* |
| Mean Right | 6.4 | 7.5 | 14.2** |
| Mean Mouth | 6.3 | 7.3 | 13.0** |
| | Treat - 1X Additive in | | |
| | treat (mixed) | | |
| Mean Left | 5.7 | 7.1 | 19.6** |
| Mean Right | 5.9 | 7.5 | 20.2** |
| Mean Mouth | 5.8 | 7.3 | 19.9** |
| | Treat - 1X Additive on | | |
| | treat (paste) | | |
| Mean Left | 5.7 | 7.1 | 19.0** |
| Mean Right | 5.8 | 7.5 | 22.0** |
| Mean Mouth | 5.8 | 7.3 | 20.5** |

TABLE 6
Day 63 Gingivitis Reduction Comparisons

| | | Control Diet | % Reduction |
|---|---|---|---|
| | Treat - no Additive | | |
| Mean Left | 0.1 | 0.2 | 33.1 |
| Mean Right | 0.1 | 0.2 | 60.0* |
| Mean Mouth | 0.1 | 0.2 | 48.8 |
| | Treat - 1X Additive in | | |
| | treat (mixed) | | |
| Mean Left | 0.1 | 0.2 | 58.2* |
| Mean Right | 0.1 | 0.2 | 42.0 |
| Mean Mouth | 0.1 | 0.2 | 48.8 |
| | Treat - 1X Additive on | | |
| | treat (paste) | | |
| Mean Left | 0.1 | 0.2 | 21.9 |
| Mean Right | 0.1 | 0.2 | 54.0 |
| Mean Mouth | 0.1 | 0.2 | 40.6 |

TABLE 7
Day 63 Calculus Reduction Comparisons

| | | Control Diet | % Reduction |
|---|---|---|---|
| | Treat - no Additive | | |
| Mean Left | 1.2 | 2.5 | 53.2** |
| Mean Right | 1.2 | 2.6 | 54.9** |
| Mean Mouth | 1.2 | 2.5 | 54.1** |
| | Treat - 1X Additive in | | |
| | treat (mixed) | | |
| Mean Left | 0.9 | 2.5 | 65.4** |
| Mean Right | 0.9 | 2.6 | 65.5** |
| Mean Mouth | 0.9 | 2.5 | 65.4** |
| | Treat - 1X Additive on | | |
| | treat (paste) | | |
| Mean Left | 0.9 | 2.5 | 61.7** |
| Mean Right | 0.9 | 2.6 | 67.5** |
| Mean Mouth | 0.9 | 2.5 | 64.6** |

TABLE 8
% of Tartar Reduction

| | | | Treat 1X |
|---|---|---|---|
| | Treat no additive | Treat 1X Additive | Additive on |
| | (treat control) | in treat (mixed) | treat (paste) |
| vs. Main Meal | 43.0 | 61.2 | 59.2 |
| (control diest) | | | |
| vs. Treat Control | 0 | 31.9 | 28.3 |

### Conclusion

The consumption of a treat without 1× Additive by Beagle dogs for 35 days resulted in a statistically significant reduction of the group mean mouth plaque, gingivitis, calculus values by 15%, 50%, and 43%, respectively, when compared to the group mean mouth plaque, gingivitis and calculus values of control dogs. The consumption of a treat without 1× Additive by Beagle dogs for 63 days resulted in a statistically significant reduction of the group mean mouth plaque and calculus values by 13% and 54%, respectively, when compared to the group mean mouth plaque and calculus values of control dogs. Gingivitis reductions were not statistically significant.

The consumption of a treat with 1× Additive in the treat by Beagle dogs for 35 days resulted in a statistically significant reduction of the group mean mouth plaque, gingivitis, calculus values by 19%, 60%, and 61%, respectively, when compared to the group mean mouth plaque, gingivitis and calculus values of control dogs. The consumption of a treat with 1× Additive in the treat by Beagle dogs for 63 days resulted in a statistically significant reduction of the group mean mouth plaque and calculus values by 20% and 65%, respectively, when compared to the group mean mouth plaque and calculus values of control dogs. Gingivitis reductions were not statistically significant.

The consumption of a treat with 1× Additive as a paste on the treat by Beagle dogs for 35 days resulted in a statistically significant reduction of the group mean mouth plaque, gingivitis, calculus values by 13%, 59%, and 59%, respectively, when compared to the group mean mouth plaque, gingivitis and calculus values of control dogs. The consumption of a treat with 1× Additive as a paste on the treat by Beagle dogs for 63 days resulted in a statistically significant reduction of the group mean mouth plaque and calculus values by 21% and 65%, respectively, when compared to the group mean mouth plaque and calculus values of control dogs. Gingivitis reductions were not statistically significant.

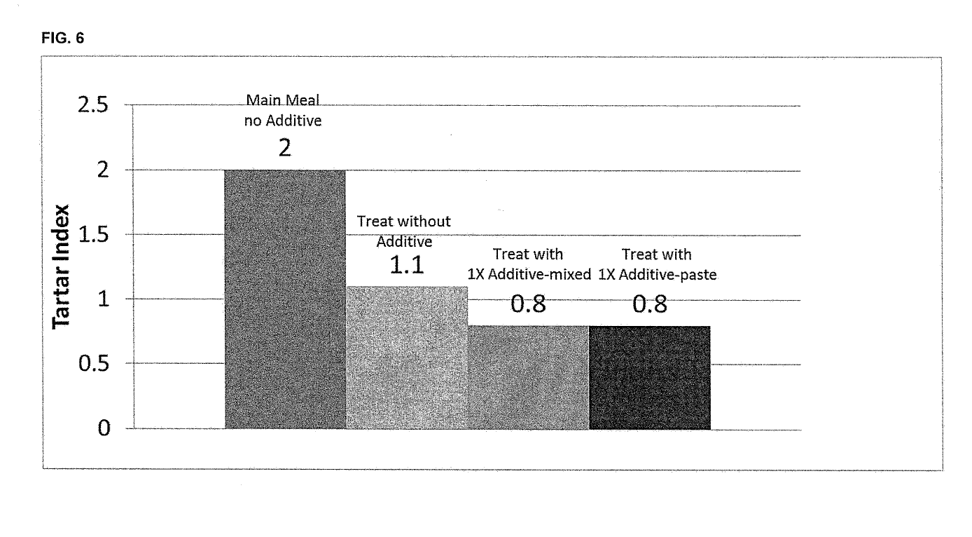

There was a significant difference of tartar index between groups fed the treat with additive compared to groups that were not fed the additives (FIG. 6 and Table 8).

### Example 3. Stability of Additive to Heat

Pet products that promote oral health by reducing plaque and calculus buildup are desirable, particularly in wet pet foods. A mixture of trisodium citrate dihydrate, disodium EDTA dihydrate and zinc chloride were exposed to high temperatures and pressures in conditions similar to retorting food. The actives were placed in 5.5 oz aluminum cans. Five additional cans were not subjected to steam retort (control) and five cans were subjected to steam over-pressure retort (retorted).

| Samples | pH | Zinc | Citric Acid | EDTA |
|---|---|---|---|---|
| Control in water | 4.80 | 435 ppm | 7.67% | 19,283 ppm |
| Retorted in water | 4.73 | 458 ppm | 7.48% | 19,434 ppm |

Results show that zinc, citric acid and EDTA levels were stable during retort with control values very similar to that of retorted values and within expected variation.

### Example 4. Retorted Food

Since our initial results showed that zinc, citric acid and EDTA levels were stable during retort, the following experiments will further confirm efficacy in wet foods using disodium EDTA, sodium citrate, and zinc citrate. Disodium EDTA can include all forms including hydrated EDTA compounds. Sodium citrate can include monosodium citrate, disodium citrate and trisodium citrate and can include hydrated compounds. Zinc citrate can include hydrated forms. In one embodiment, the additives can be trisodium citrate, disodium EDTA and zinc citrate trihydrate.

A wet food is produced with the additives to ensure that the food matrix does not affect stability during retort. Canned products are produced with the additives in either the loaf, chunk or gravy portion of the food. Concentrations of the additives range from 1× to 5×. Stability of the additives is determine in the gravy and chunk portion of the food.

Palatability is tested of canned food containing the additives to ensure animal acceptance. From this test, a concentration of additives can be determined for testing for oral health efficacy.

The effect of the additives is determined in canned food on oral health parameters, for example, gingival scores, plaque, calculus and tartar, similar to Examples 1 and 2. A standard VOHC feeding test evaluating the accumulation of plaque and tartar in a clean mouth over a period of 28 or 56 days is performed. This is done by the following comparisons in feeding trial: 1) Control wet food (no additives), 2) Wet food with additives in chunks, 3) Wet food with additives in gravy, 4) Wet food with additives in loaf. Results are expected to be significantly similar to the results from Examples 1 and 2.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of reducing or preventing conversion of plaque to calculus, the method comprising orally administering to a companion animal a composition comprising one or more chelating agents selected from the group consisting of sodium citrate, potassium citrate, zinc citrate trihydrate, disodium ethylenediaminetetraacetic acid (EDTA), tetrasodium EDTA, ethylene glycol tetraacetic acid (EGTA), diethylenetriaminepentaacetic acid (DTPA), ethylenediamine-N,N′-bis(2-hydroxyphenylacetic acid) (EDDHA), iminodiacetic acid (IDA), trans-I,2-diaminocyclohexane-N,N,N′,N′-tetraacetate (CDTA), hydroxyethyenediaminetriacetic acid (HEDTA), 2-hydroxyethyliminodiacetic acid (HEIDA), and nitrilotriacetic acid (NTA), the one or more chelating agents are present in a total amount of at least about 6.0 g/L of the composition.

2. The method of claim 1 wherein the total amount of the one or more chelating agents is about 0.01% to about 5.0% of the composition.

3. The method of claim 1 wherein the chelating agent comprises (i) a first chelating agent selected from the group consisting of sodium citrate, potassium citrate, zinc citrate and mixtures thereof and (ii) a second chelating agent selected from the group consisting of disodium ethylenediaminetetraacetic acid (EDTA), tetrasodium EDTA, ethylene glycol tetraacetic acid (EGTA), diethylenetriaminepentaacetic acid (DTPA), ethylenediamine-N,N′-bis(2-hydroxyphenylacetic acid) (EDDHA), iminodiacetic acid (IDA), trans-I,2-diaminocyclohexane-N,N,N′,N′-tetraacetate (CDTA), hydroxyethyenediaminetriacetic acid (HEDTA), 2-hydroxyethyliminodiacetic acid (HEIDA), nitrilotriacetic acid (NTA) and mixtures thereof.

4. The method of claim 3, wherein the first chelating agent comprises sodium citrate.

5. The method of claim 3, wherein the first chelating agent is present in an amount from about 0.01% to about 5.0% of the composition.

6. The method of claim 3, wherein the second chelating agent comprises disodium EDTA.

7. The method of claim 3, wherein the second chelating agent is present in an amount from about 0.01% to about 1.0% of the composition.

8. The method of claim 1, further comprising a zinc salt, wherein the zinc salt is selected from the group consisting of zinc chloride, zinc lactate, zinc gluconate, and mixtures thereof.

9. The method of claim 8, wherein the zinc salt comprises zinc chloride.

10. The method of claim 8, wherein the zinc salt is present in an amount from about 0.001% to about 5.0% of the composition.

11. The method of claim 1, wherein the composition further comprises a preservative.

12. The method of claim 11, wherein the preservative comprises a benzoate salt.

13. The method of claim 1, wherein the composition further comprises citric acid.

14. The method of claim 1, wherein the composition is administered in an orally acceptable carrier selected from the group consisting of water, a foam, a pet treat, an extruded kibble, a coating of a pet food, an edible film, a wet pet food, a retorted pet food, and a retorted canned pet food.

15. The method of claim 1, wherein the composition is administered without increasing the daily caloric intake of the companion animal relative to a time period comprising at least one week immediately prior to the administering.

16. A comestible composition comprising one or more chelating agents selected from the group consisting of sodium citrate, potassium citrate, zinc citrate, disodium ethylenediaminetetraacetic acid (EDTA), tetrasodium EDTA, ethylene glycol tetraacetic acid (EGTA), diethylenetriaminepentaacetic acid (DTPA), ethylenediamine-N,N′-bis(2-hydroxyphenylacetic acid) (EDDHA), iminodiacetic acid (IDA), trans-I,2-diaminocyclohexane-N,N,N′,N′-tetraacetate (CDTA), hydroxyethyenediaminetriacetic acid (HEDTA), 2-hydroxyethyliminodiacetic acid (HEIDA), and nitrilotriacetic acid (NTA), the one or more chelating agents are present in a total amount of at least about 6.0 g/L of the comestible composition, and the comestible composition further comprises a zinc salt.

17. The comestible composition of claim 16, wherein the one or more chelating agents comprise trisodium citrate, disodium EDTA, and zinc citrate in an amount from about 0.01% to about 5.0% of the composition.

18. The comestible composition of claim 17, wherein the composition comprises an orally acceptable carrier selected from the group consisting of a foam, a pet treat, an extruded kibble, a coating of a pet food, an edible film, a wet pet food, a retorted pet food, and a retorted canned pet food.

19. The comestible composition of claim 18, wherein the orally acceptable carrier is a pet treat, an extruded kibble, a wet pet food, a retorted pet food, or a retorted canned pet food.
